(19) United States
(12) Patent Application Publication
(10) Pub. No.: US 2026/0176700 A1
(43) Pub. Date: Jun. 25, 2026

(54) TOXICITY MARKER FOR 5-FLUOROURACIL

(71) Applicants: Oxford University Innovation Limited, Oxford (GB); The Universtiy of Birmingham, Birmingham (GB)

(72) Inventors: Claire Palles, Birmingham (GB); David Kerr, Oxford (GB); Rachel Kerr, Oxford (GB)

(21) Appl. No.: 19/126,119

(22) PCT Filed: Nov. 3, 2023
(86) PCT No.: PCT/GB2023/052878

§ 371 Date: Apr. 30, 2025

(30) Foreign Application Priority Data
Nov. 3, 2022 (GB) 2216376.0

(51) Int. Cl. C12Q 1/6886; A61K 31/704; A61K 31/7068; A61K 31/7105; A61K 45/06

(52) U.S. Cl. CPC C12Q 1/6886; A61K 31/704; A61K 31/7068; A61K 31/7105; A61K 45/06; C12Q 2600/118; C12Q 2600/156

(57) ABSTRACT

The invention relates to an assay. More specifically, the invention relates to an assay for predicting cardiovascular toxicity of a chemotherapeutic agent.

The invention relates to an assay. More specifically, the invention relates to an assay for predicting cardiovascular toxicity of a chemotherapeutic agent.

5-flurouracil (5FU) is a fluoropyrimidine antimetabolite drug that is used in the adjuvant chemotherapy setting where it has been shown to increase survival of patients with colorectal, gastric and breast cancers. Capecitabine (pentyl [1-(3,4-dihydroxy-5-methyltetrahydrofuran-2-yl)-5-fluoro-2-oxo-1H-pyrimidin-4-yl]carbamate) is an oral prodrug of 5FU that is widely used in chemotherapy of many cancer types. However, use of 5FU or prodrugs thereof (e.g. capecitabine) is limited by concomitant toxicities that arise during treatment in some patients. Among these concomitant toxicities, cardiovascular toxicity is a rare but potentially life threatening side effect. After anthracyclines, fluoropyrimidines are the most common cause of chemotherapy-related cardiovascular toxicity (Sorrentino, M. F., et al. Cardiol J, 2012. 19(5):453-8). Cardio-oncology is an emerging field of great importance as a growing number of cancer patients present with existing cardiovascular comorbidities.

NCI Common Toxicity Criteria for Adverse Events (CTCAE) version 3.0:

CTCAE v.3.0

| GRADE | MEANING |
|---|---|
| 1 | Mild Adverse Event |
| 2 | Moderate Adverse Event |
| 3 | Severe Adverse Event |
| 4 | Life-threatening or disabling Adverse Event |
| 5 | Death related to Adverse Event |

Dihydropyrimidine dehydrogenase (DPD) is the rate-limiting enzyme involved in the breakdown of 5FU (Diasio R B, et al. Clin Pharmacokinet. (1989); 16(4):215-37). Four variants mapping to the DPYD gene which encodes DPD have been identified by the Clinical Pharmacogenomics Implementation Consortium (CPIC) as having sufficient evidence to be used in clinical practice to screen for complete or partial DPD deficiency to guide subsequent 5FU chemotherapy dose modification (Amstutz U, et al. Clin Pharmacol Ther. (2018); 103(2):210-6). However, the current strategy of testing for these four variants has low sensitivity, probably owing to failure to test for other toxicity associated variants mapping to within and outside the fluoropyrimidine metabolism pathway. Patients with germline DPYD gene variants which result in a reduction in DPD activity have a greatly increased risk of 5FU-related toxicity of any type, but only 2-5% of patients with reduced DPD activity experience cardiotoxicity.

Capecitabine is activated to 5FU via a three-step enzymatic process via carboxylesterase (CES), cytidine deaminase (CDA) and thymidine phosphorylase (TYMP) (Lam S W, et al. Cancer Treat Rev (2016); 50:9-22). Several pharmacogenetic studies have reported capecitabine toxicity associated variants in 5FU metabolising enzymes thymidylate synthase (TYMS) and DPYD, capecitabine activating enzymes CDA and CES2 and methylenetetrahydrofolate reductase (MTHFR) (Lam S W, et al. Cancer Treat Rev (2016); 50:9-22). However, the reported findings so far have been inconsistent between studies possibly because of small sample sizes, heterogeneity in sample populations and different drug regimens (Lam S W, et al. Cancer Treat Rev (2016); 50:9-22).

There is currently no way to identify patients who are at risk of developing 5FU-induced cardiovascular toxicity.

There is accordingly an urgent and unmet need for genetic markers that can be used to identify patients who are at risk of 5FU-induced cardiovascular toxicity. There is likewise an unmet need for assays that are predictive of 5FU-induced cardiovascular toxicity, and for kits and reagents for performing said assays.

The invention addresses the above needs. In particular, the inventors have discovered genetic biomarkers and related assays that are predictive of 5FU-induced cardiovascular toxicity. In more detail, the inventors have identified 14 genetic markers that are highly predictive of 5FU-induced cardiovascular toxicity. These genetic markers (rs4904753, rs57742598, rs72213987, rs28548905, rs10148571, rs4900072, rs4900071, rs4904758, rs4904755, rs533803730, rs34420600, rs10137483, rs17224411, and rs61990142) share the same allele frequency and are in the same genomic region. rs4904753 is used herein as a representative marker but any of the genetic markers listed above may be used instead. As used herein, functionally equivalent variants of rs4904753 include the genetic markers rs57742598, rs72213987, rs28548905, rs10148571, rs4900072, rs4900071, rs4904758, rs4904755, rs533803730, rs34420600, rs10137483, rs17224411, and rs61990142.

Advantageously, patients who have been identified as candidates for 5FU chemotherapy can be screened for the presence of rs4904753 or a functionally equivalent variant thereof, whereby the presence of a toxicity associated allele of at least one of these genetic markers indicates an increased risk of 5FU-induced cardiovascular toxicity. Patients who are identified as being at an increased risk 5FU-induced cardiovascular toxicity may be monitored for signs of cardiovascular toxicity during treatment with 5FU or a prodrug thereof (e.g. capecitabine), or may be treated with an alternative chemotherapeutic regimen that does not require 5FU administration.

The invention provides a method of screening for risk of 5FU-induced cardiovascular toxicity in a patient, comprising screening the patient for the presence of rs4904753 or a functionally equivalent variant thereof, wherein: (i) the presence of rs4904753 or a functionally equivalent variant thereof indicates an increased risk of developing 5FU-induced cardiovascular toxicity compared to a patient which does not possess rs4904753 or a functionally equivalent variant thereof; and (ii) the absence of rs4904753 or a functionally equivalent variant thereof indicates no increased risk or a decreased risk of developing 5FU-induced cardiovascular toxicity compared to a patient which possesses rs4904753 or a functionally equivalent variant thereof. In some embodiments, the functionally equivalent variant of rs4904753 is selected from rs57742598, rs72213987, rs28548905, rs10148571, rs4900072, rs4900071, rs4904758, rs4904755, rs533803730, rs34420600, rs10137483, rs17224411, and rs61990142.

Herein, it will be understood that ‘administration of 5FU’ includes administration of prodrug(s) of 5FU (such as with capecitabine); and that ‘treatment with 5FU’ includes treatment with prodrug(s) of 5FU (such as with capecitabine). Similarly, ‘5FU-induced cardiovascular toxicity’ includes cardiovascular toxicity induced by prodrug(s) of 5FU (e.g. capecitabine); ‘chemotherapy with 5FU’ includes chemotherapy with prodrug(s) of 5FU (e.g. capecitabine); ‘5FU administration’ includes administration of prodrug(s) of 5FU (e.g. capecitabine), and the like.

A positive result (i.e. the presence of rs4904753 or a functionally equivalent variant thereof) indicates an increased risk of developing cardiovascular toxicity following or during treatment with 5FU (e.g. following or during treatment with capecitabine). A negative result (i.e. absence of rs4904753 or a functionally equivalent variant thereof) indicates no increased risk or a decreased risk of developing cardiovascular toxicity following or during treatment with 5FU (e.g. following or during treatment with capecitabine).

Patients carrying two copies of the toxicity associated allele of rs4904753 or two copies of the toxicity associated allele of a functionally equivalent variant thereof are at an extremely high risk of 5FU-induced cardiovascular toxicity. For example, patients carrying two copies of the T allele of rs4904753 are at an extremely high risk of 5FU-induced cardiovascular toxicity (odds ratio (OR) 21.78 (95% CI 8.15-58.24), p-value=8.37×10⁻¹⁰). Thus, in some embodiments, (i) the presence of two copies of the toxicity associated allele of rs4904753 or two copies of the toxicity associated allele of a functionally equivalent variant thereof indicates an increased risk of developing 5FU-induced cardiovascular toxicity compared to a patient which possesses one or no copies of the toxicity associated allele of rs4904753 or one or no copies of a functionally equivalent variant thereof; and (ii) the presence of one or no toxicity associated alleles of rs4904753 or one or no copies of a functionally equivalent variant thereof indicates a decreased risk of developing 5FU-induced cardiovascular toxicity compared to a patient which possesses two toxicity associated alleles of rs4904753 or two toxicity associated alleles of a functionally equivalent variant thereof. Toxicity associated alleles are provided in Table 1.

Screening for two copies of the T allele of rs4904753 has surprisingly been found to provide a sensitivity of 79.2% and a specificity of 91.7% for detection of patients at risk of 5FU-induced cardiovascular toxicity (e.g. capecitabine-induced cardiovascular toxicity). Screening for a functionally equivalent variant of rs4904753 advantageously provides the same sensitivity and specificity as testing for rs4904753 because these genetic markers are inherited together.

In one embodiment, the patient is a cancer patient. In one embodiment, the patient has a solid tumour cancer. In one embodiment, the patient has colorectal cancer (CRC), breast cancer, or gastrointestinal cancer.

In one embodiment, the patient has been identified as a candidate for chemotherapy with 5FU or a prodrug thereof, optionally wherein the patient has been identified as a candidate for chemotherapy with capecitabine.

In one embodiment, the patient is undergoing chemotherapy with 5FU, optionally wherein the patient is undergoing chemotherapy with capecitabine.

In one embodiment, the screening is carried out on a fluid sample from the patient. In one embodiment, the fluid sample is a saliva, blood serum or plasma sample.

In one embodiment, the screening is carried out on a solid sample from the patient. In one embodiment, the solid sample is from a biopsy.

In one embodiment, in the absence of rs4904753 or a functionally equivalent variant thereof, the method further comprises identifying the patient as a candidate for chemotherapy with 5FU or a prodrug thereof, optionally wherein the prodrug is capecitabine.

In one embodiment, in the presence of rs4904753 or a functionally equivalent variant thereof, the patient is identified as a candidate for chemotherapy which does not comprise administration of 5FU or a prodrug thereof (e.g. capecitabine). In one embodiment, in the presence of two copies of the toxicity associated allele of rs4904753 or two copies of the toxicity associated allele of a functionally equivalent variant thereof, the patient is identified as a candidate for chemotherapy which does not comprise administration of 5FU or a prodrug thereof (e.g. capecitabine).

Clinical situations may arise in which 5FU or a prodrug thereof is administered to a patient possessing rs4904753 or a functionally equivalent variant thereof. In such cases, the present invention can help inform clinicians on the dose of 5FU administered to the patient. In one embodiment, in the presence of rs4904753 or a functionally equivalent variant thereof (e.g. in the presence of two copies of the toxicity associated allele of rs4904753 or two copies of the toxicity associated allele of a functionally equivalent variant thereof), the patient is identified as a candidate for chemotherapy with 5FU or a prodrug thereof, wherein said chemotherapy comprises administration of a lower starting doses of 5FU or prodrug than would typically be administered. The skilled person is well aware of the typical administration doses of 5FU and prodrugs thereof, and can identify suitable lower doses. For example, a patient having two copies of the toxicity associated allele of rs4904753 or two copies of the toxicity associated allele of a functionally equivalent variant thereof may be administered capecitabine at a dose of 625 mg/m² twice daily for 14 days or at a dose of 1250 mg/m² once daily for 14 days, at a dose of 500 mg/m² twice daily for 14 days, or at a dose of 1000 mg/m² once daily for 14 days.

In clinical situations in which 5FU or a prodrug thereof is administered to a patient possessing rs4904753 or a functionally equivalent variant thereof, the present invention can also help inform clinicians on the type or frequency of patient monitoring. In one embodiment, in the presence of rs4904753 or a functionally equivalent variant thereof, the method further comprises monitoring the patient for one or more symptoms of cardiovascular toxicity during and/or after treatment with 5FU, or a prodrug thereof (e.g. capecitabine).

As used herein, “5FU-induced cardiovascular toxicity” embraces a range of conditions affecting the heart and/or blood vessels of a patient. Typical 5FU-induced cardiovascular toxicity symptoms include chest pain, such as atypical chest pain and angina, and acute coronary syndromes such as myocardial infarction. Cardiovascular toxicity is a well-known side effect of 5FU therapy, and methods for diagnosing cardiovascular toxicity are known in the art. The terms “cardiovascular toxicity”, “cardiac toxicity”, and “cardiotoxicity” are used interchangeably herein. Cardiovascular toxicity may be graded in accordance with the NCI Common Toxicity Criteria for Adverse Events (CTCAE) version 3.0. In some embodiments, the 5FU-induced cardiovascular toxicity risk may be high-grade toxicity, i.e. grade 3+.

The ‘rs’ number refers to the dbSNP ID. dbSNP (“Single Nucleotide Polymorphism database”) is a public-domain archive for human single nucleotide variations, microsatellites, and small-scale insertions. dbSNP is developed and hosted by the National Center for Biotechnology Information in collaboration with the National Human Genome Research Institute. rs4904753 and the functionally equivalent variants described herein map to the open reading frame gene C14orf159 recently annotated as D-glutamate cyclase (DGLUCY). dbSNP annotates rs4904753 at chr14: 91153616 (GRCh38.p13).

In some embodiments, the presence of two copies of the toxicity associated allele of rs4904753 or two copies of the toxicity associated allele of a functionally equivalent variant thereof (reported in the forward orientation) indicates an increased risk of developing 5FU-induced cardiovascular toxicity compared to a patient which does not possess two copies of the toxicity associated allele of rs4904753 or two copies of the toxicity associated allele of a functionally equivalent variant thereof.

In one embodiment, the invention provides a method comprising: isolating a sample from a patient and performing an assay on the sample to detect rs4904753 or a functionally equivalent variant thereof. In one embodiment, the invention provides a method comprising detecting in a sample rs4904753 or a functionally equivalent variant thereof. In one embodiment, the invention provides a method comprising: isolating a sample from a patient and performing an assay on the sample to detect a toxicity associated allele of rs4904753 or a toxicity associated allele of a functionally equivalent variant thereof. In one embodiment, the invention provides a method comprising detecting in a sample a toxicity associated allele of rs4904753 or a toxicity associated allele of a functionally equivalent variant thereof.

The sample may be a fluid sample, such as a saliva, blood, serum or plasma sample. The sample may be a solid sample, such as a sample from a biopsy.

In one embodiment, the method comprises diagnosing the patient as at risk of developing 5FU-induced cardiovascular toxicity when the presence of rs4904753 or a functionally equivalent variant thereof is detected in the sample. In one embodiment, the method comprises diagnosing the patient as at risk of developing 5FU-induced cardiovascular toxicity when the presence of two toxicity associated alleles of rs4904753 or two toxicity associated alleles of a functionally equivalent variant thereof are detected in the sample.

In one embodiment, the method comprises diagnosing the patient as at risk of developing 5FU-induced cardiovascular toxicity when the presence of rs4904753 or a functionally equivalent variant thereof is detected in the sample; and administering to the patient a chemotherapeutic agent that does not comprise 5FU or a prodrug thereof (e.g. capecitabine). In one embodiment, the method comprises diagnosing the patient as at risk of developing 5FU-induced cardiovascular toxicity when the presence of two toxicity associated alleles of rs4904753 or two toxicity associated alleles of a functionally equivalent variant thereof is detected in the sample; and administering to the patient a chemotherapeutic agent that does not comprise 5FU or a prodrug thereof.

In one embodiment, the method further comprises administering to the patient a chemotherapeutic agent that does not comprise 5FU or a prodrug thereof (e.g. capecitabine), when the presence of rs4904753 or a functionally equivalent variant thereof is detected in the sample. In one embodiment, the method further comprises administering to the patient a chemotherapeutic agent that does not comprise 5FU or a prodrug thereof (e.g. capecitabine), when the presence of two toxicity associated alleles of rs4904753 or two toxicity associated alleles of a functionally equivalent variant thereof are detected in the sample.

In one embodiment, the invention provides a method comprising obtaining a sample from a patient; and exposing the sample to a reagent capable of detecting in the sample the presence of rs4904753 or a functionally equivalent variant thereof. Suitable reagents are known in the art and may comprise primers and/or probe(s) specific for rs4904753 or a functionally equivalent variant thereof. In one embodiment, the invention provides a method comprising obtaining a sample from a patient; and exposing the sample to a reagent capable of detecting in the sample the presence of a toxicity associated allele of rs4904753 or a toxicity associated allele of a functionally equivalent variant thereof.

The invention also provides a composition for detecting the presence of rs4904753 or a functionally equivalent variant thereof in a sample obtained from a patient, comprising: (a) the sample; and (b) means for amplifying rs4904753 or a functionally equivalent variant thereof. The invention also provides a composition for detecting the presence of a toxicity associated allele of rs4904753 or a toxicity associated allele of a functionally equivalent variant thereof in a sample obtained from a patient, comprising: (a) the sample; and (b) means for amplifying a toxicity associated allele of rs4904753 or a toxicity associated allele of a functionally equivalent variant thereof.

The patient may be a cancer patient. The patient may have a solid tumour cancer such as colorectal cancer (CRC), breast cancer, or gastrointestinal cancer. The patient may be undergoing (or have undergone) chemotherapy with 5FU. Alternatively, the method may be carried out on a patient who has not yet undergone chemotherapy with 5FU, such as a patient who has been identified as a candidate for chemotherapy with 5FU. Chemotherapy with 5FU includes chemotherapy with 5FU prodrugs, such as capecitabine.

Chemotherapy with 5FU may be a 5FU monotherapy, such as capecitabine monotherapy. The term “chemotherapy with 5FU” however includes any therapy based on 5FU either alone or in combination with one or more other agents, e.g. FOLFOX (folinic acid, 5FU and oxaliplatin), XELOX (capecitabine plus oxaliplatin), or FOLFIRI (calcium folinate, 5FU and irinotecan).

Screening for polymorphisms with equivalent functional effects to rs4904753 is also included within the scope of this invention. Such variants may be screened for using available gene sequencing or direct functional assays as are known in the technical field.

The method of the invention permits close monitoring of a patient in which rs4904753 or a functionally equivalent variant thereof is present and who is therefore identified as being at increased risk of 5FU-induced cardiovascular toxicity. For example, the method of the invention permits close monitoring of a patient having two toxicity associated allele(s) of rs4904753 or two toxicity associated alleles of a functionally equivalent variant thereof and who is therefore identified as being at risk of 5FU-induced cardiovascular toxicity. The method of the invention may accordingly include a step of monitoring the patient for symptoms of 5FU-induced cardiovascular toxicity in the event of a positive result, i.e. where rs4904753 or a functionally equivalent variant thereof is detected (e.g. where two toxicity associated alleles of rs4904753 or two toxicity associated alleles of a functionally equivalent variant thereof are detected).

The invention also provides one or more reagents capable of detecting the presence of rs4904753 or a functionally equivalent variant thereof e.g. in a sample obtained from a patient, for use in the method of the invention. Suitable reagents may include primers and/or probes specific to rs4904753 or a functionally equivalent variant thereof, e.g. a toxicity associated allele of rs4904753 or a toxicity associated allele of a functionally equivalent variant thereof.

The above-mentioned reagents may be present in a kit. Accordingly, the invention provides a kit comprising one or more of the above-mentioned reagents capable of detecting the presence of rs4904753 or a functionally equivalent variant thereof.

Screening for the presence of rs4904753 or a functionally equivalent variant thereof may comprise sequencing methods known in the art such as direct sequencing or PCR. Screening for the presence of rs4904753 or a functionally equivalent variant thereof may comprise hybridisation-based methods known in the art, e.g. using allele-specific probes (such as T allele specific probes).

### FIGURES

FIG. 1: Manhattan plot of genome wide association study (GWAS) of cardiovascular toxicity in QUASAR 2. Summary statistics from GWAS of QUASAR 2 were plotted using FUMA. The dashed line indicates the genome wide significance threshold 5×10⁻⁸. 14 common SNPs all marking a single genomic location reached this statistical threshold. These SNPs map to an open reading frame gene C14orf159 recently annotated as D-glutamate cyclase (DGLUCY) because of the similarity between this human gene and a bacterial aspartate/glutamate/hydantoin racemase. DGLUCY is expressed at high levels in ventricular and atrial cardiomyocytes and has been shown to localise to the mitochondria of these cells (Ariyoshi, M., et al. Sci Rep, 2017. 7:43911). The locus identified is also in linkage disequilibrium (LD) with a locus shown to be associated with heart rate and all-cause mortality (Eppinga, R. N., et al. Nat Genet, 2016. 48(12):1557-1563).

FIG. 2: Receiver operator curve analysis to determine sensitivity and specificity of testing for two copies of the toxicity associated T allele of SNP rs4904753 for predicting those at risk of cardiovascular toxicity following treatment with capecitabine. QUASAR 2 data were used to test the sensitivity and specificity of a test consisting of the number of copies of the toxicity associated allele of rs4904753.

FIG. 3: Transcript level analysis of RNA sequencing and genotyping data from the GTEX project identifies the cardiovascular toxicity associated variant as an expression quantitative trait locus for the DGLUCY gene. Expression data was plotted by genotype. The cardiovascular toxicity associated T allele is statistically significantly associated with expression of the major DGLUCY transcript in tissue samples from the left ventricle (data shown), aorta, atrial appendage and skeletal muscle.

### EXAMPLES

The invention will be further clarified by the following examples, which are intended to be purely exemplary of the invention and are in no way limiting.

### Methods

### Data

QUASAR 2, a randomised controlled trial of capecitabine+bevacizumab has been described previously (Kerr, R. S., et al. Lancet Oncol, 2016. 17(11):1543-1557; and Rosmarin, D., et al. Gut, 2015. 64(1):111-20). Briefly, participants were stage II/stage III colorectal cancer patients with performance status 0/1 and normal renal function. Patients were randomly assigned (1:1) to receive eight 3-week cycles of oral capecitabine alone (1250 mg/m² twice daily for 14 days followed by a break for 7 days) or the same regimen of oral capecitabine plus 16 cycles of 7.5 mg/kg bevacizumab by intravenous infusion over 90 min on day 1 of each cycle. All patients consented for use of biological samples and medical records in research. Cardiovascular toxicity was confirmed by two independent clinicians reviewing patient case report forms.

### Genotyping

QUASAR 2 samples have been genotyped using whole genome SNP tagging arrays and imputed using 1000 genomes and UK10K merged imputation panel as described in Rosmarin, D., et al., Gut, 2015. 64(1):111-20. The lead cardiovascular toxicity associated SNP, rs4904753, was directly genotyped in 759 samples to confirm the accuracy of the imputation of this SNP. This confirmatory genotyping was performed using a KASP (LGC) competitive allele specific PCR. Three positive control samples and a negative control were included on each genotype plate. The genotypes generated conformed to hardy Weinberg equilibrium and 100% concordance was observed when repeat genotyping a randomly selected 5% of samples.

### Statistical Analysis

Pre-imputation and post imputation quality control have been previously described (Rosmarin, D., et al., Gut, 2015. 64(1):111-20). The two arms of QUASAR2 were analysed separately for associations with cardiovascular toxicity and the results were combined by meta-analysis using GWAMA (Magi, R. and A. P. Morris, GWAMA: software for genome-wide association meta-analysis. BMC Bioinformatics, 2010. 11:288). SNPs and indels were evaluated for association with cardiovascular toxicity. Forest plots were generated using R package “meta”. Manhattan plots were created using LocusZoom.

### Results

### Genome Wide Association Study of Capecitabine-Induced Cardiovascular Toxicity Identifies a Novel Genetic Locus

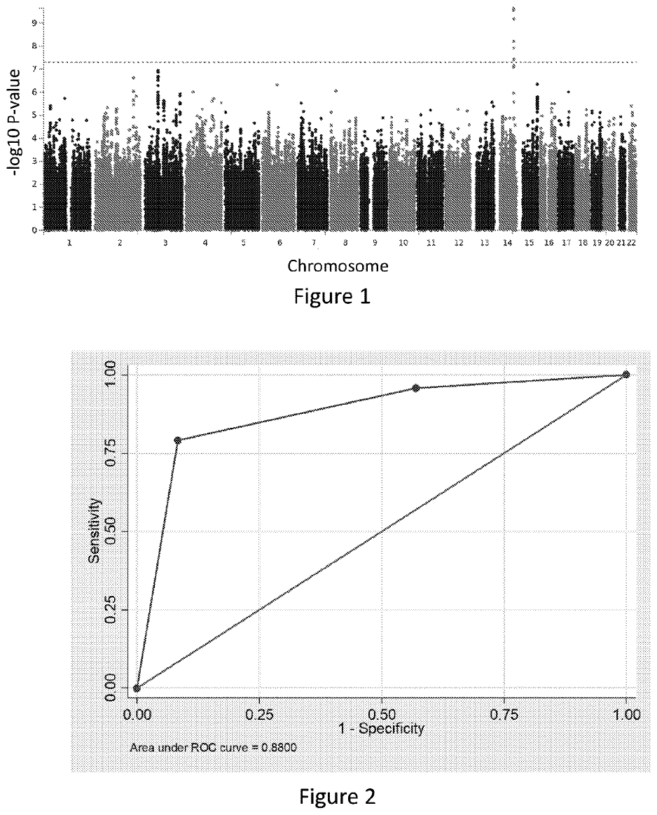

3% of colorectal cancer patients administered with capecitabine as part of the QUASAR 2 clinical trial experienced cardiovascular related adverse events. The most commonly described events were cardiac chest pain and angina. The inventors performed a genome wide association study to identify genetic markers associated with cardiovascular adverse events under an additive genetic model. Given the rarity of cardiovascular toxicity, variants with a minor allele frequency (MAF)<10% were filtered out. One signal was identified at genome wide significance on chromosome 14, mapping to the gene D-glutamate cyclase (DGLUCY, previously C14orf159) (FIG. 1). This gene has a domain with homology to the aspartate/glutamate/hydrontoin racemase of Thermovirga lienii. It localises to the heart mitochondria and has been found to have D-glutamate cyclase activity (Ariyoshi, M., et al. Sci Rep, 2017. 7:43911). 14 genetic markers reached the genome wide significance threshold of 5×10⁻⁸ (FIG. 1, Table 1). Of these, rs4904753 reached the highest significance and was selected as a representative marker.

rs4904753 was imputed in QUASAR 2 (see methods) with an INFO score of 0.81. Whilst this indicates high confidence imputation, the inventors also directly genotyped the SNP in 759/930 QUASAR 2 samples where DNA was available. Very similar results were obtained using the genotype results and the concordance between imputed and genotyped calls was 0.87. Results from the direct genotyping were used in subsequent analyses.

Under an additive genetic model the T allele of rs4904753 is associated with an increased risk of cardiovascular toxicity (OR 9.08 (95% CI 4.38-18.82), p-value=2.97×10⁻⁹). The four DPYD variants currently suggested by the Clinical Pharmacogenetics Implementation Consortium (CPIC) for testing prior to administering fluoropyrimidines have similar magnitudes of effect on risk of developing grade 3-4 toxicities (Meulendijks, D., et al. Lancet Oncol, 2015. 16(16):1639-50; and Rosmarin, D., et al. J Clin Oncol, 2014. 32(10):1031-9). Interestingly, whilst the DPYD variants are associated with grade 3/grade 4 haematological and gastrointestinal toxicities in QUASAR 2 samples they are not associated with risk of cardiovascular toxicity (combined test of the four variants and an association with cardiovascular toxicity: p-value=0.07).

Clinical Utility of this Novel Marker of Cardiovascular Toxicity

96% (23/24) of QUASAR 2 participants who developed cardiovascular toxicity carried one or more rs4904753 T alleles with 79% (19/24) of these patients carrying two copies of the rs4904753 T allele. By comparison only 8% (61/735) of QUASAR 2 participants who did not experience cardiovascular toxicity carried two copies of the T allele. FIG. 2 shows the sensitivity and specificity of testing for rs4904753 in QUASAR 2 for the detection of those at risk of cardiovascular toxicity following treatment with capecitabine. At a cut point of 2, the test yields a sensitivity of 79.2% and a specificity of 91.7% for the detection of those at risk of cardiovascular toxicity.

When rs4904753 was analysed under a recessive genetic model, those carrying two copies of the T allele were identified as being at an extremely high risk of cardiovascular toxicity (OR 21.78 (95% CI 8.15-58.24), p-value=8.37×10⁻¹⁰). This result is highly statistically significant and testing for two copies of rs4904753 yields very good sensitivity and specificity (FIG. 2).

### Discussion

The inventors have discovered the first genetic locus associated with cardiovascular toxicity following treatment with the 5FU prodrug capecitabine. The inventors have demonstrated that testing for rs4904753 or a functionally equivalent variant thereof provides high sensitivity and specificity for the detection of those at risk of developing cardiovascular toxicity. These genetic markers provide important clinical utility for screening for patients prior to the administration of 5FU containing regimens, particularly when administration of capecitabine is contemplated.

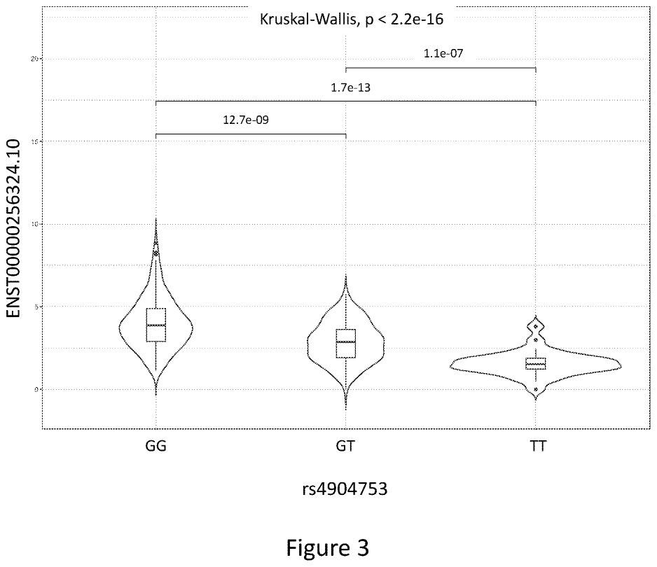

The mechanism by which rs4904753 or a functionally equivalent variant thereof acts to increase the risk of cardiovascular toxicity following treatment with capecitabine is not clear. Knowledge of the mode of action of the SNP is not required however for it to be useful as a clinical biomarker for identifying those at risk of cardiovascular toxicity. Whilst rs4904753 maps to intron 3 of transcript variant 1 (uc001xyv.2) of DGLUCY, it is possible that this intronic region is a regulatory element for an alternative gene. The inventors investigated whether rs4904753 or linked markers were associated with levels of gene expression using data from the Genotype-Tissue Expression project (GTEX)(https://gtexportal.org). rs4904753 was associated with levels of expression of DGLUCY in multiple tissue types but not with expression of any other gene (Genotype-Tissue Expression GTEX data). At the gene level the toxicity associated T allele was associated with increased levels of expression of DGLUCY in skeletal muscle (P=2×10⁻⁸) but decreased levels in heart atrial appendage (P=1.3×10⁻⁶). Genotyping and transcript level expression data was downloaded from GTEX (see methods) to enable a transcript level analysis. The most abundant transcript across skeletal muscle and all heart tissues evaluated was ENST00000256324.10. The toxicity allele was consistently associated with lower levels of expression of this transcript across all examined tissues (FIG. 3). New expression data from blood samples from 31,684 individuals (Vosa, U., et al. Nat Genet, 2021. 53(9):1300-1310) identified that rs4904753 is associated with increased expression of RPS6KA5 (p-value=7.37×10⁻²²) and with decreased expression of DGLUCY (p-value=1.44×10⁻¹⁸).

rs4904753 and the functionally equivalent variant rs4900072 are also identified as splice QTLs (sQTL) in GTEX data in most tissue types including skeletal muscle and heart tissues. rs4900072 has a missense impact on most transcripts but a splice donor impact on three transcripts: ENST00000520328.1, ENST00000523816.1 and ENST00000428926.2 (all other toxicity associated variants were intronic or did not alter the amino acid sequence (synonymous changes)). The missense impact of rs4900072 is predicted (by SIFT and polyphen) to be benign in most transcripts including the most abundant transcript and would be predicted to lead to a retained intron in the three transcripts listed above where the SNP maps to a splice donor site. The relevance of these transcripts relative to the most abundant transcripts remains to be determined but increased expression of ENST00000428926 in TT versus GG/GT samples in multiple tissue types was noted.

eQTL/sQTL data suggest DGLUCY and RPS6KA5 as possible targets of the cardiovascular toxicity associated SNPs. Chromatin conformation data from cardiomyocytes shows chromatin looping between the cardiovascular toxicity associated SNP and the promoter regions of both RPS6KA5 and GPR68 (Montefiori, L. E., et al. Elife, 2018. 7).

In summary, the inventors have identified rs4904753 and functionally equivalent variants thereof as genetic markers that are highly predictive of 5FU-induced cardiac toxicity. These genetic markers can be used to screen patients prior to treatment with 5FU, and those who are identified as being at an increased risk of 5FU-induced cardiac toxicity may be treated via a different method, e.g. by chemotherapeutic methods that do not require administration of 5FU or a prodrug thereof.

TABLE 1
Genetic markers with MAF > 10% and associated with cardiovascular toxicity at P < 5 × 10⁻⁸

| | | | toxicity | | | | | | |
|---|---|---|---|---|---|---|---|---|---|
| Positional based ID | | | associated | other | | | | | |
| (hg19 genome build) | rs ID | Consequence | allele | allele | MAF | Beta | SE | P | N |
| 14:91647993_CTTTT_C | rs57742598 | intron_variant | C | CTTTT | 0.268198 | 2.339603 | 0.36546 | 1.59E−10 | 930 |
| 14:91623533_GGATA_G | rs72213987 | intron_variant | G | GGATA | 0.338891 | 2.12861 | 0.335325 | 2.25E−10 | 930 |
| 14:91619960_G_T | rs4904753 | intron_variant | T | G | 0.338838 | 2.126483 | 0.335355 | 2.36E−10 | 930 |
| 14:91622583_G_A | rs28548905 | intron_variant | A | G | 0.339866 | 2.118759 | 0.334929 | 2.60E−10 | 930 |
| 14:91621154_A_G | rs10148571 | intron_variant | G | A | 0.339452 | 2.120308 | 0.335474 | 2.69E−10 | 930 |
| 14:91636532_C_T | rs4900072 | missense_variant | T | C | 0.339493 | 2.12323 | 0.335983 | 2.71E−10 | 930 |
| 14:91636395_C_T | rs4900071 | synonymous_variant | T | C | 0.340024 | 2.119719 | 0.335725 | 2.81E−10 | 930 |
| 14:91643173_T_A | rs4904758 | intron_variant | A | T | 0.339114 | 2.122377 | 0.336212 | 2.83E−10 | 930 |
| 14:91631360_G_A | rs4904755 | intron_variant | A | G | 0.348048 | 2.100697 | 0.340015 | 6.68E−10 | 930 |
| 14:91648025_C_CT | rs533803730 | intron_variant | CT | C | 0.309431 | 2.044114 | 0.350997 | 5.91E−09 | 930 |
| 14:91653239_C_CA | rs34420600 | intron_variant | CA | C | 0.309798 | 2.016006 | 0.346633 | 6.19E−09 | 930 |
| 14:91660044_C_T | rs10137483 | intron_variant | T | C | 0.281895 | 1.896528 | 0.332544 | 1.21E−08 | 930 |
| 14:91659769_G_A | rs17224411 | intron_variant | A | G | 0.280332 | 1.855538 | 0.336799 | 3.69E−08 | 930 |
| 14:91656057_A_G | rs61990142 | intron_variant | G | A | 0.280581 | 1.850283 | 0.336502 | 3.92E−08 | 930 |

1. A method of screening for risk of 5-fluorouracil (5FU)-induced cardiovascular toxicity in a patient, comprising screening the patient for the presence of rs4904753 or a functionally equivalent variant thereof, wherein:
(i) the presence of rs4904753 or a functionally equivalent variant thereof indicates an increased risk of developing 5FU-induced cardiovascular toxicity compared to a patient which does not possess rs4904753 or a functionally equivalent variant thereof; and
(ii) the absence of rs4904753 or a functionally equivalent variant thereof indicates no increased risk or a decreased risk of developing 5FU-induced cardiovascular toxicity compared to a patient which possesses rs4904753 or a functionally equivalent variant thereof.

2. The method according to claim 1, wherein the functionally equivalent variant is selected from rs57742598, rs72213987, rs28548905, rs10148571, rs4900072, rs4900071, rs4904758, rs4904755, rs533803730, rs34420600, rs10137483, rs17224411, and rs61990142.

3. The method according to claim 1 or claim 2, wherein the patient is a cancer patient.

4. The method according to claim 3, wherein the patient has a solid tumour cancer.

5. The method according to claim 3 or claim 4, wherein the patient has colorectal cancer (CRC), breast cancer, or gastrointestinal cancer.

6. The method according to any one of the preceding claims, wherein the patient has been identified as a candidate for chemotherapy with 5FU, optionally wherein the patient has been identified as a candidate for chemotherapy with capecitabine.

7. The method according to any one of the preceding claims, wherein the patient is undergoing chemotherapy with 5FU, optionally wherein the patient is undergoing chemotherapy with capecitabine.

8. The method according to any one of the preceding claims, wherein the screening is carried out on a fluid sample from the patient.

9. The method according to claim 8, wherein the fluid sample is a saliva, blood serum or plasma sample.

10. The method according to any one of the preceding claims, wherein the screening is carried out on a solid sample from the patient.

11. The method according to claim 10, wherein the solid sample is from a biopsy.

12. The method according to any one of the preceding claims, wherein in the absence of rs4904753 or a functionally equivalent variant thereof, the method further comprises identifying the patient as a candidate for chemotherapy with 5FU or a prodrug thereof, optionally wherein the prodrug is capecitabine.

13. The method according to any one of the preceding claims, wherein in the presence of rs4904753 or a functionally equivalent variant thereof, the patient is identified as a candidate for chemotherapy which does not comprise administration of 5FU or a prodrug thereof, such as capecitabine.

14. The method according to any one of the preceding claims, wherein in the presence of rs4904753 or a functionally equivalent variant thereof, the method further comprises monitoring the patient for one or more symptoms of cardiovascular toxicity during and/or after treatment with 5FU, or a prodrug thereof.
